(19) United States
(12) Patent Application Publication
(10) Pub. No.: US 2026/0115056 A1
(43) Pub. Date: Apr. 30, 2026

(54) SANITARY PRODUCT WITH THREE-DIMENSIONAL CUFFS

(71) Applicant: Beijing Beishute Maternity and Child Articles Co., Ltd., Beijing (CN)

(72) Inventors: Lin CAO, Beijing (CN); Qiuhong LI, Beijing (CN); Qian WANG, Beijing (CN); Min GAO, Beijing (CN); Jie ZHANG, Beijing (CN)

(73) Assignee: Beijing Beishute Maternity and Child Articles Co., Ltd., Beijing (CN)

(21) Appl. No.: 19/470,840

(22) PCT Filed: Jul. 30, 2024
(86) PCT No.: PCT/CN2024/108334

§ 371 Date: Sep. 30, 2025

(30) Foreign Application Priority Data
Aug. 2, 2023 (CN) 202310965782.1

(51) Int. Cl. A61F 13/475; A61F 13/15; A61F 13/47; A61F 13/511; A61F 13/515; A61F 13/56; A61L 15/30; A61L 15/58

(52) U.S. Cl. CPC A61F 13/4752; A61F 13/15203; A61F 13/4704; A61F 13/51121; A61F 13/515; A61F 13/5611; A61L 15/30; A61L 15/58; A61F 2013/1556

(57) ABSTRACT

A sanitary product with three-dimensional cuffs includes an absorbent body and three-dimensional cuffs. The three-dimensional cuff is composed of a water-repellent thermal-bonded nonwoven fabric, one or more rubber bands, and a lining fabric. The water-repellent thermal-bonded nonwoven fabric is strip-shaped; its inner side edge is bent downward and outward to form a bent portion with an internal space. The outer bottom surface of the bent portion is adhered to the surface of the absorbent body, and the outer side edge of the water-repellent thermal-bonded nonwoven fabric is adhered to the outer edge of the absorbent body. The one or more rubber bands are adhered between the upper surface of the lining fabric and the top surface of the internal space of the bent portion via rubber band glue, and the lower surface of the lining fabric is separated from the bottom surface of the internal space of the bent portion.

### CROSS REFERENCE TO THE RELATED APPLICATIONS

This application is the national phase entry of International Application No. PCT/CN2024/108334, filed on Jul. 30, 2024, which is based upon and claims priority to Chinese Patent Application No. 202310965782.1, filed on Aug. 2, 2023, the entire contents of which are incorporated herein by reference.

### TECHNICAL FIELD

The present disclosure relates to the field of disposable sanitary products, and in particular to a sanitary product with three-dimensional cuffs.

### BACKGROUND

For sanitary absorbent products used for urinary incontinence or menstrual periods, side leakage is a common problem. The gush of discharged body fluids may temporarily submerge the surface of the absorbent product, thereby leaking over the side edges. To solve this problem, absorbent products are provided with three-dimensional cuffs along their lateral edges in various ways to prevent body fluid leakage at the sides. However, existing products have poor actual side leakage prevention effects during specific use. Moreover, the elastic materials such as rubber bands at the edges of existing three-dimensional cuffs are relatively rough, which will rub against the wearer's skin, resulting in poor comfort.

As disclosed in the prior art patent application CN201180031683.1, there is an absorbent article with improved leg cuffs. The absorbent article includes a topsheet, a backsheet joined to the topsheet, and an absorbent core positioned between the topsheet and the backsheet. The absorbent core may have longitudinal edges and end edges, and the absorbent article may be provided with leg cuffs extending along each longitudinal edge of the absorbent core. The leg cuffs can be in a raised position and a flattened position; when in the raised position, the cross-sectional area of the leg cuffs is approximately quadrilateral. The cross-sectional area is defined by an upper surface, an inner barrier, an outer barrier, and a lower surface. However, the design of such quadrilateral leg cuffs is highly material-wasteful, as the width of the fabric on both sides is 30% larger than that of conventional three-dimensional cuffs. Moreover, during use, the quadrilateral structure is prone to being pressed together, failing to function as a leakage-proof barrier. Additionally, since the elastic components are located on the upper surface of the leg cuffs and come into direct contact with the wearer's body, discomfort still persists.

Furthermore, the prior art patent application CN201921488398.2 discloses a care pad with a three-dimensional cuff structure. The care pad includes a care pad body and a three-dimensional cuff structure. The three-dimensional cuff structure includes an upper nonwoven layer, a lower nonwoven layer, a left nonwoven layer, and a right nonwoven layer. The lower nonwoven layer is parallel to the care pad body and connected to the care pad body at its lower part. An elastic layer is arranged in parallel below the upper nonwoven layer, so that the upper nonwoven layer, lower nonwoven layer, left nonwoven layer, and right nonwoven layer form a cylinder-like shape parallel to the care pad body. However, the elastic layer described in this utility model is an elastic fabric with longitudinal elasticity, which is difficult to source and challenging to implement in the production process. Furthermore, this three-dimensional cuff structure is prone to collapsing, resulting in poor side leakage prevention performance.

It can be seen that the aforementioned existing side leakage-proof sanitary products obviously still have inconveniences and defects in terms of structure and actual production, and thus urgently require further improvement. In the current industry, it is an urgent improvement target to create a new sanitary product with three-dimensional cuffs, which forms cylindrical three-dimensional cuffs through the cooperative effect of rubber band, water-repellent thermal-bonded nonwoven fabric, and lining fabric. This sanitary product not only improves the wearer's comfort but also truly achieves the goal of preventing side leakage. Additionally, it is easy to implement in the production process and low in cost.

### SUMMARY

The technical problem to be solved by the present disclosure is to provide a sanitary product with three-dimensional cuffs, which forms cylindrical three-dimensional cuffs through the cooperative effect of rubber band, water-repellent thermal-bonded nonwoven fabric, and lining fabric. This sanitary product not only improves the wearer's comfort but also truly achieves the goal of preventing side leakage. Additionally, it is easy to implement in the production process and low in cost, thereby overcoming the shortcomings of existing side leakage-proof sanitary products.

To solve the above technical problem, the present disclosure provides a sanitary product with three-dimensional cuffs. The sanitary product includes a strip-shaped absorbent body and three-dimensional cuffs respectively arranged on both sides of the absorbent body in a length direction. The three-dimensional cuff is composed of a water-repellent thermal-bonded nonwoven fabric, one or more rubber bands, and a lining fabric. The water-repellent thermal-bonded nonwoven fabric is strip-shaped, and an inner side edge of the water-repellent thermal-bonded nonwoven fabric is bent downward and outward to form a bent portion with an internal space. An outer bottom surface of the bent portion is adhered to a surface of the absorbent body, and an outer side edge of the water-repellent thermal-bonded nonwoven fabric is adhered to an outer edge of the absorbent body. The lining fabric is horizontally arranged in the internal space of the bent portion. The one or more rubber bands are adhered between an upper surface of the lining fabric and a top surface of the internal space of the corresponding bent portion via rubber band glue. A lower surface of the lining fabric is separated from a bottom surface of the internal space of the bent portion; thus, the lining fabric is supported in the internal space of the bent portion by an elastic force of the one or more rubber bands to form cylindrical three-dimensional cuffs.

In a further improvement, the lining fabric is made of spunbond+meltblown+spunbond (SMS) nonwoven fabric with a basis weight of 25-40 gsm.

In a further improvement, a length of the lining fabric is equal to a length of the water-repellent thermal-bonded nonwoven fabric.

In a further improvement, a length of the one or more rubber bands is less than the length of the water-repellent thermal-bonded nonwoven fabric, and the one or more rubber bands are located in a middle section of the water-repellent thermal-bonded nonwoven fabric.

In a further improvement, two parallel rubber bands are provided in each bent portion.

In a further improvement, in a bonding area between the outer bottom surface of the bent portion and the surface of the absorbent body, inner edges of the bonding areas at both ends of the absorbent body are closer to a center of the absorbent body than edges of the bonding area in a middle section of the absorbent body.

In a further improvement, the outer bottom surface of the bent portion is adhered to the surfaces at both ends of the absorbent body via two adhesive strips, and the outer bottom surface of the bent portion is adhered to the surface of the middle section of the absorbent body via one adhesive strip.

In a further improvement, the absorbent body sequentially includes, from top to bottom, a surface layer, a diversion layer, toilet paper, an absorption layer, toilet paper, a bottom film, and release paper. These layers are adhered together with glue, and an annular groove is further pressed on the absorbent body between the two three-dimensional cuffs.

In a further improvement, the annular groove is designed into an asymmetric structure with a wider front end and a narrower rear end.

In a further improvement, an elliptical groove is further pressed at a front end inside the annular groove, the diversion layer is arranged inside the elliptical groove, and the diversion layer adopts a blue-core diversion layer.

After adopting such a design, the present disclosure has at least the following advantages:

The sanitary product with three-dimensional cuffs of the present disclosure, which has its outer side enclosed by the water-repellent thermal-bonded nonwoven fabric and its inner side equipped with the one or more rubber bands and the lining fabric in a cooperative manner, can cleverly ensure the supporting height of the three-dimensional cuffs. This enables the three-dimensional cuffs to not only have a good water-repellent function, but also, due to the softness and comfort of the water-repellent thermal-bonded nonwoven fabric and the arc-shaped outer circumference of the cylindrical three-dimensional cuffs, effectively reduce skin friction, making the product more comfortable for the wearer when in use, with excellent leakage-proof effect and significantly improved comfort. In addition, the materials used are easily available, the processing technology is simple, the production can be completed online on a single production line, and the cost is low.

### BRIEF DESCRIPTION OF THE DRAWINGS

The above is merely an overview of the technical solution of the present disclosure. To understand the technical means of the present disclosure more clearly, the present disclosure will be further described in detail below with reference to the drawings and specific implementations.

FIG. 1 is a front view schematic diagram of the structure of the sanitary product with three-dimensional cuffs of the present disclosure.

FIG. 2 is a three-dimensional structure schematic diagram of the sanitary product with three-dimensional cuffs of the present disclosure.

FIG. 3 is a cross-sectional view taken along line A-A in FIG. 1.

FIG. 4 is a cross-sectional view taken along line B-B in FIG. 1.

### DETAILED DESCRIPTION OF THE EMBODIMENTS

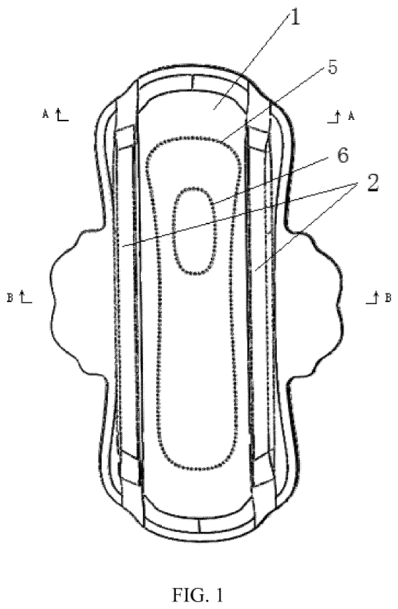

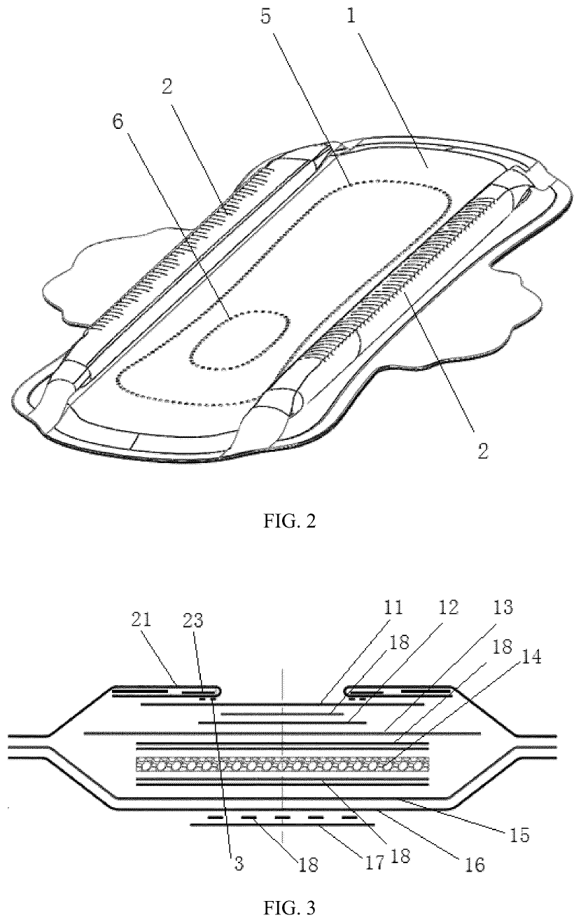

As shown in the FIG. 1 to FIG. 4, the sanitary product with three-dimensional cuffs of this embodiment includes a strip-shaped absorbent body 1, with three-dimensional cuffs 2 respectively arranged on both sides of the absorbent body 1 in the length direction.

In this embodiment, the absorbent body 1 sequentially includes, from top to bottom, a surface layer 11, a diversion layer 12, toilet paper 13, an absorption layer 14, toilet paper 15, a bottom film 16, and release paper 17. These layers are adhered together with glue 18 to form an absorbent body with good absorption effect. Of course, the absorbent body 1 can also adopt other existing structures of absorbent bodies.

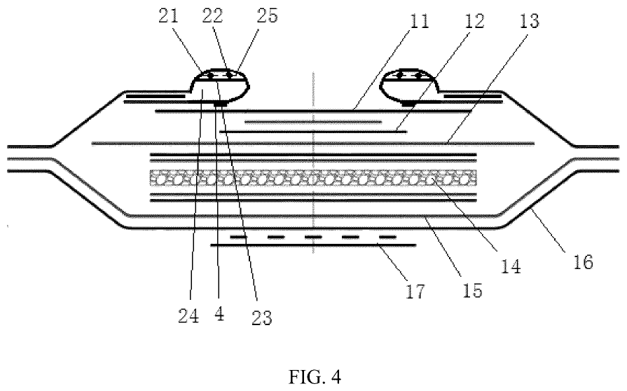

In this embodiment, the three-dimensional cuff 2 is composed of a water-repellent thermal-bonded nonwoven fabric 21, one or more rubber bands 22, and a lining fabric 23. The water-repellent thermal-bonded nonwoven fabric 21 is strip-shaped, and an inner side edge of the water-repellent thermal-bonded nonwoven fabric 21 is bent downward and outward and then adhered to the lower surface of the water-repellent thermal-bonded nonwoven fabric 21 to form a bent portion with an internal space 24. The outer bottom surface of the bent portion is adhered to the surface of the absorbent body 1. The outer side edge of the water-repellent thermal-bonded nonwoven fabric 21 is adhered with the outer edge of the absorbent body 1. The lining fabric 23 is horizontally arranged in the internal space 24 of the bent portion, the one or more rubber bands 22 are adhered between the upper surface of the lining fabric 23 and the top surface of the internal space 24 of the corresponding bent portion via rubber band glue 25, the lower surface of the lining fabric 23 is separated from the bottom surface of the internal space 24 of the bent portion; thus, the lining fabric 23 is supported in the internal space 24 of the bent portion by the elastic force of the one or more rubber bands 22 to form a cylindrical three-dimensional cuff, as shown in FIG. 4. The one or more rubber bands 22 are in a stretched state during processing and shrinks in a natural state, cooperating with the lining fabric to form the cylindrical support of the bent portion. Since the outer side of the cylindrical three-dimensional cuff 2 is arc-shaped, there is no edge that rubs against the body, which greatly improves comfort.

In this embodiment, the lining fabric 23 is made of SMS nonwoven fabric with a basis weight of 25-40 gsm. The SMS nonwoven fabric has a certain degree of stiffness, which can better ensure that the bent portion forms a cylindrical three-dimensional cuff 2.

Specifically, the length of the lining fabric 23 is equal to the length of the water-repellent thermal-bonded nonwoven fabric 21. The length of the one or more rubber bands 22 is less than the length of the water-repellent thermal-bonded nonwoven fabric 21, and the one or more rubber bands 22 are located in the middle section of the water-repellent thermal-bonded nonwoven fabric 21; that is, the height of the cylindrical three-dimensional cuff is high in the middle section of the absorbent body 1, and gradually decreases at both ends of the absorbent body 1, forming a flush bond with both ends of the absorbent body 1, which conforms to the characteristics of the human body when using the product, with low middle and high ends, and has a good side leakage prevention performance.

In a preferred embodiment, two parallel rubber bands 22 are provided in each bent portion. In the bonding area between the outer bottom surface of the bent portion and the surface of the absorbent body 1, the inner edges of the bonding areas at both ends of the absorbent body 1 are closer to the center of the absorbent body 1 than the edges of the bonding area in the middle section of the absorbent body 1; that is, the three-dimensional cuffs 2 fit gradually at both ends of the absorbent body 1, while the support degree of the three-dimensional cuffs 2 gradually increases in the middle section of the absorbent body 1, forming a cuff structure that meets the requirement for preventing side leakage. Moreover, this cuff structure is not prone to collapse and has a good support effect.

Specifically, the outer bottom surface of the bent portion is adhered to the surfaces at both ends of the absorbent body 1 via two adhesive strips 3, and the outer bottom surface of the bent portion is adhered to the surface of the middle section of the absorbent body 1 via one adhesive strip 4. Of course, an alternative implementation is to widen the width of the adhesive strips arranged at both ends of the bent portion and narrow the width of the adhesive strip arranged in the middle section of the bent portion.

Additionally, in the sanitary product with three-dimensional cuffs of this embodiment, an annular groove 5 is further pressed on the absorbent body 1 between the two three-dimensional cuffs 2. The annular groove 5 is designed into an asymmetric structure with a wider front end and a narrower rear end, which makes it more in line with the human physiological structure and more conducive to the precise positioning, infiltration and absorption of body fluids.

In this embodiment, an elliptical groove 6 is further pressed at the front end inside the annular groove 5, and the diversion layer 12 is arranged inside the elliptical groove 6. The diversion layer 12 adopts a blue-core diversion layer to enhance the diversion effect.

The absorbent body 1 can be a product with wings or without wings, which is set according to actual needs.

The sanitary product with three-dimensional cuffs of this embodiment features an innovative structural design, easily available materials, and can be manufactured online on a single production line. It avoids the use of elastic fabrics that are difficult to source and apply, with a simple production process and low cost.

The present disclosure is a sanitary product with comfortable and elastic three-dimensional cuffs. Since the nonwoven fabric on the outer side of the three-dimensional cuffs is a water-repellent thermal-bonded nonwoven fabric, it not only has a water-repellent function but also is soft and comfortable, providing a comfortable feel when in contact with the human body. Additionally, by bending the water-repellent thermal-bonded nonwoven fabric and arranging a lining fabric and one or more rubber bands inside the bent portion, the top of the three-dimensional cuffs can be well supported, thereby achieving an effective leakage-proof effect. Moreover, due to the arc-shaped outer circumference of the cylindrical three-dimensional cuffs, friction against the skin is effectively reduced, making the product more comfortable for the wearer when in use.

The above description is merely a preferred embodiment of the present disclosure and does not impose any formal restriction on the present disclosure. Any simple modifications, equivalent changes, or modifications made by those skilled in the art based on the technical content disclosed above shall all fall within the protection scope of the present disclosure.

What is claimed is:

1. A sanitary product with three-dimensional cuffs, comprising a strip-shaped absorbent body, wherein the three-dimensional cuffs are respectively arranged on both sides of the strip-shaped absorbent body in a length direction, and each of the three-dimensional cuffs is composed of a water-repellent thermal-bonded nonwoven fabric, one or more rubber bands, and a lining fabric; the water-repellent thermal-bonded nonwoven fabric is strip-shaped, and an inner side edge of the water-repellent thermal-bonded nonwoven fabric is bent downward and outward to form a bent portion with an internal space; an outer bottom surface of the bent portion is adhered to a surface of the strip-shaped absorbent body, and an outer side edge of the water-repellent thermal-bonded nonwoven fabric is adhered to an outer edge of the strip-shaped absorbent body; the lining fabric is horizontally arranged in the internal space of the bent portion, the one or more rubber bands are adhered between an upper surface of the lining fabric and a top surface of the internal space of the bent portion via a rubber band glue, and a lower surface of the lining fabric is separated from a bottom surface of the internal space of the bent portion; and thus, the lining fabric is supported in the internal space of the bent portion by an elastic force of the one or more rubber bands to form cylindrical three-dimensional cuffs;
the lining fabric is made of spunbond+meltblown+spunbond (SMS) nonwoven fabric with a basis weight of 25-40 gsm, and a length of the lining fabric is equal to a length of the water-repellent thermal-bonded nonwoven fabric;
a length of each of the one or more rubber bands is less than the length of the water-repellent thermal-bonded nonwoven fabric, and the one or more rubber bands are located in a middle section of the water-repellent thermal-bonded nonwoven fabric;
in a bonding area between the outer bottom surface of the bent portion and the surface of the strip-shaped absorbent body, inner edges of bonding areas at both ends of the strip-shaped absorbent body are closer to a center of the strip-shaped absorbent body than an edge of a bonding area in a middle section of the strip-shaped absorbent body; and
the outer bottom surface of the bent portion is adhered to surfaces at the both ends of the strip-shaped absorbent body via two adhesive strips, and the outer bottom surface of the bent portion is adhered to a surface of the middle section of the strip-shaped absorbent body via one adhesive strip.

2. The sanitary product with the three-dimensional cuffs according to claim 1, wherein portion the one or more rubber bands comprise two rubber bands provided in the bent portion in parallel

3. The sanitary product with the three-dimensional cuffs according to claim 1, wherein the strip-shaped absorbent body sequentially comprises, from top to bottom, a surface layer, a diversion layer, a first toilet paper, an absorption layer, a second toilet paper, a bottom film, and a release paper, wherein the surface layer, the diversion layer, the first toilet paper, the absorption layer, the second toilet paper, the bottom film, and the release paper are adhered together with a glue, and an annular groove is further pressed on the strip-shaped absorbent body between the three-dimensional cuffs.

4. The sanitary product with the three-dimensional cuffs according to claim 3, wherein the annular groove is designed into an asymmetric structure with a wider front end and a narrower rear end.

5. The sanitary product with the three-dimensional cuffs according to claim 4, wherein an elliptical groove is further pressed at a front end inside the annular groove, the diversion layer is arranged inside the elliptical groove, and the diversion layer adopts a blue-core diversion layer.

6-10. (canceled)

11. The sanitary product with the three-dimensional cuffs according to claim 2, wherein the strip-shaped absorbent body sequentially comprises, from top to bottom, a surface layer, a diversion layer, a first toilet paper, an absorption layer, a second toilet paper, a bottom film, and a release paper, wherein the surface layer, the diversion layer, the first toilet paper, the absorption layer, the second toilet paper, the bottom film, and the release paper are adhered together with a glue, and an annular groove is further pressed on the strip-shaped absorbent body between the three-dimensional cuffs.
